(19) United States
(12) Patent Application Publication
(10) Pub. No.: US 2010/0124311 A1
(43) Pub. Date: May 20, 2010

(54) Tomographic image capturing apparatus

(75) Inventors: Jun Enomoto, Kanagawa-ken (JP); Hirofumi Sawada, Minami-ashigara-shi (JP); Sadato Akahori, Odawara-shi (JP); Eiichi Kanagawa, Minami-ashigara-shi (JP); Tomoyoshi Nishimura, Tokyo (JP); Yasunori Ohta, Yokohama-shi (JP); Noriaki Ida, Minami-ashigara-shi (JP)

Correspondence Address: AKERMAN SENTERFITT, 8100 BOONE BOULEVARD, SUITE 700, VIENNA, VA, 22182-2683, US

(73) Assignee: FUJIFILM Corporation, Tokyo (JP)

(21) Appl. No.: 12/591,194

(22) Filed: Nov. 12, 2009

(30) Foreign Application Priority Data
Nov. 18, 2008 (JP) 2008-294680

(51) Int. Cl. A61B 6/03

(52) U.S. Cl. 378/9

(57) ABSTRACT

A tomographic image capturing apparatus includes a plurality of radiation sources movable along a predetermined path, for applying respective radiation to a subject at different angles and at different times while moving along the predetermined path, a dose adjuster for adjusting the radiation sources to equalize doses of the radiation emitted from the radiation sources, a radiation detector for detecting radiation transmitted through the subject while the radiation sources move along the predetermined path, and converting the detected radiation into respective items of radiographic image information, and a tomographic image generator for generating a tomographic image of the subject based on the items of radiographic image information converted from the radiation and detected by the radiation detector.

### CROSS-REFERENCE TO RELATED APPLICATION

This application is based upon and claims the benefit of priority from Patent Application No. 2008-294680 filed on Nov.18, 2008, in the Japan Patent Office, of which the contents are incorporated herein by reference.

### BACKGROUND OF THE INVENTION

1. Field of the Invention

The present invention relates to a tomographic image capturing apparatus for capturing a tomographic image of a subject using a plurality of radiation sources.

2. Description of the Related Art

Primarily in the field of medicine, tomographic image capturing apparatus have been used for producing tomographic images of subjects such as a patient by applying radiation to the subject at different angles, so as to obtain different items of radiographic image information, and thereafter processing the items of radiographic image information. The tomographic image capturing apparatus may include a CT (Computerized Tomography) apparatus and a tomosynthesis apparatus, which are known in the art.

The CT apparatus includes a radiation source and a radiation detector, which are disposed in confronting relation to each other. In operation, while the radiation source and the radiation detector revolve around a subject to be imaged, the radiation source emits radiation, and the radiation detector detects radiation that has passed through the subject. Radiographic image information, which is generated based on the radiation detected by the radiation detector, is processed in order to produce a tomographic image of the subject, along a plane in which the radiation source and the radiation detector revolve. The tomosynthesis apparatus also has a radiation source and a radiation detector, which are disposed in confronting relation to each other. In operation, while the radiation source and the radiation detector move relatively along a subject to be imaged, the radiation source emits radiation, and the radiation detector detects radiation that has passed through the subject. A tomographic image of the subject, which has been taken along the direction in which the radiation source and the radiation detector move, is generated based on radiation detected by the radiation detector. The CT apparatus tends to be large and costly due to the fact that the radiation source and the radiation detector must revolve around the subject. The tomosynthesis apparatus is more compact and less expensive than a CT apparatus, because the radiation source and the radiation detector move within a smaller range, due to the limited angle within which radiation is applied.

Such tomographic image capturing apparatus include a radiation source and a radiation detector, which are set in motion for enabling capturing of tomographic images. In particular, since the tomographic image capturing apparatus must produce a large number of items of radiographic image information in order to obtain a high resolution tomographic image, a long period of time is consumed before the image capturing process is completed.

Japanese Laid-Open Patent Publication No. 2004-188200 discloses a tomosynthesis apparatus having two movable radiation sources, which are spaced a predetermined distance from each other in a direction perpendicular to the direction in which the radiation sources move. The radiation sources are alternately turned on and off repeatedly, i.e., one of the radiation sources is energized to emit radiation and the other radiation source is deenergized, while the radiation sources are moved simultaneously. The tomosynthesis apparatus, with the radiation sources thus controlled, is capable of acquiring a large number of items of radiographic image information within a short time period.

However, using a plurality of radiation sources to acquire a large number of items of radiographic image information is subject to disadvantages. If the radiation sources emit different doses of radiation, then the resolution of the tomographic image, which is reconstructed from the radiographic image information, tends to be low, even though the period of time required to produce the tomographic image is short.

### SUMMARY OF THE INVENTION

An object of the present invention is to provide a tomographic image capturing apparatus, which can shorten the time required to capture a tomographic image, and which is capable of generating a high resolution tomographic image.

According to the present invention, there is provided a tomographic image capturing apparatus comprising a plurality of radiation sources movable along a predetermined path, for applying respective radiation to a subject at different angles and at different times, a dose adjuster for adjusting the radiation sources to equalize doses of the radiation emitted from the radiation sources, a radiation detector for detecting radiation transmitted through the subject while the radiation sources move along the predetermined path, and converting the detected radiation into respective items of radiographic image information, and a tomographic image generator for generating a tomographic image of the subject based on the items of radiographic image information converted from the radiation detected by the radiation detector.

According to the present invention, there also is provided a tomographic image capturing apparatus comprising a plurality of radiation sources movable along a predetermined path, for applying respective radiation to a subject at different angles and at different times, a radiation detector for detecting radiation transmitted through the subject while the radiation sources move along the predetermined path, and converting the detected radiation into respective items of radiographic image information, an image corrector for correcting the items of radiographic image information in order to cancel out a difference between doses of the radiation emitted from the radiation sources, and a tomographic image generator for generating a tomographic image of the subject based on the items of radiographic image information which have been corrected by the image corrector.

The tomographic image capturing apparatus acquires radiographic image information using plural radiation sources, which are adjusted to equalize the radiation doses emitted therefrom. Therefore, the tomographic image capturing apparatus can complete the image capturing process within a short period of time, and can generate a high resolution tomographic image based on the radiographic image information. The tomographic image capturing apparatus is capable of generating a high resolution tomographic image from a number of different items of radiographic image information, which are acquired using the radiation sources.

Instead of adjusting the doses of radiation emitted from the radiation sources, the acquired items of radiographic image information may be corrected in order to cancel out differences between the doses, for thereby generating a high resolution tomographic image.

The above and other objects, features, and advantages of the present invention will become more apparent from the following description when taken in conjunction with the accompanying drawings in which preferred embodiments of the present invention are shown by way of illustrative example.

### BRIEF DESCRIPTION OF THE DRAWINGS

FIG. 1 is a schematic view, partially in block form, showing a tomosynthesis apparatus, which forms a tomographic image capturing apparatus according to a first embodiment of the present invention;

FIG. 2 is a block diagram showing a modification of the tomosynthesis apparatus according to the first embodiment;

FIG. 3 is a schematic view, partially in block form, showing a tomosynthesis apparatus, which forms a tomographic image capturing apparatus according to a second embodiment of the present invention; and

FIG. 4 is an explanatory drawing showing another mode concerning movement directions of the radiation sources that make up the tomosynthesis apparatus.

### DESCRIPTION OF THE PREFERRED EMBODIMENTS

Like or corresponding parts are denoted by like or corresponding reference characters throughout the views.

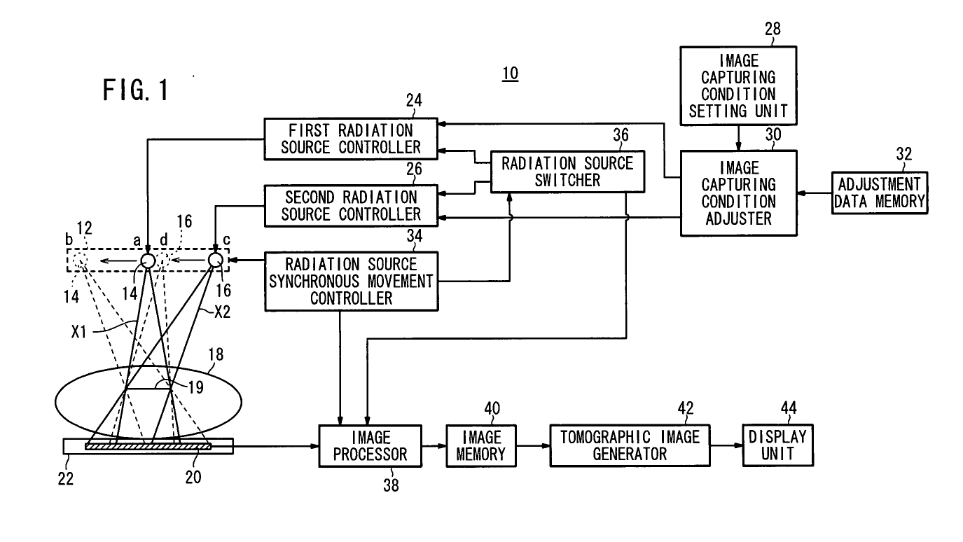

FIG. 1 schematically shows, partially in block form, a tomosynthesis apparatus 10, which serves as a tomographic image capturing apparatus according to a first embodiment of the present invention.

As shown in FIG. 1, the tomosynthesis apparatus 10 includes two radiation sources 14, 16, which can be moved by a moving mechanism 12 in the same directions, as indicated by the arrows, and a radiation detector 20 for detecting radiation X1, X2 emitted respectively from the radiation sources 14, 16 and which is transmitted through a subject 18 to be imaged, and for converting the detected radiation X1, X2 into respective items of radiographic image information. The tomosynthesis apparatus 10 generates a tomographic image of a region 19 of the subject 18 from the items of radiographic image information, when the radiation sources 14, 16 respectively apply radiation X1, X2 to the subject 18 while moving within respective ranges a to b and c to d above the subject 18. The radiation detector 20 may comprise a solid-state detector, such as an FPD (Flat Panel Detector), for converting the radiation X1, X2 into respective electric signals. The radiation detector 20 is housed in an image capturing base 22, with the subject 18 being positioned on the image capturing base 22.

The radiation sources 14, 16 are controlled respectively by a first radiation source controller 24 and a second radiation source controller 26, so as to emit radiation X1 and X2 respectively at predetermined doses. The first radiation source controller 24 and the second radiation source controller 26 control the radiation sources 14, 16 according to image capturing conditions, which are set by an image capturing condition setting unit 28 and adjusted by an image capturing condition adjuster (dose adjuster) 30. The image capturing conditions include tube voltages and tube currents that are supplied to the radiation sources 14, 16, and irradiation times for the radiation X1, X2. Doses of the radiation X1, X2 are determined by the products (mAs values) of the tube currents (mA) and the irradiation times (s).

An adjustment data memory 32 is connected to the image capturing condition adjuster 30. The adjustment data memory 32 stores adjustment data for adjusting the image capturing conditions that are set by the image capturing condition setting unit 28. The adjustment data comprise data for adjusting image capturing conditions in order to equalize the doses of radiation X1, X2 emitted from the respective radiation sources 14, 16. The adjustment data may be established as a coefficient for adjusting the mAs value of either one of the radiation sources 14, 16. The adjustment data are generated based on the doses of radiation X1, X2 emitted respectively from the radiation sources 14, 16, which are controlled according to given image capturing conditions, and are stored in the adjustment data memory 32.

The moving mechanism 12 is controlled by a radiation source synchronous movement controller 34 for moving the radiation sources 14, 16 synchronously in respective directions from positions a and c toward positions b and d. A radiation source switcher 36 is connected to the first radiation source controller 24 and the second radiation source controller 26. The radiation source switcher 36 controls the first radiation source controller 24 and the second radiation source controller 26 alternately, to turn on and off the radiation sources 14, 16 according to positional information of the radiation sources 14, 16, which is acquired from the radiation source synchronous movement controller 34. More specifically, the radiation source switcher 36 controls the first radiation source controller 24 and the second radiation source controller 26, in order to repeat a process of turning on the radiation source 14 to emit radiation X1 while simultaneously turning off the radiation source 16, and then turning off the radiation source 14 while simultaneously turning on the radiation source 16 to emit radiation X2.

An image processor 38 is connected to the radiation detector 20. The image processor 38 processes items of radiographic image information acquired from the radiation detector 20 when the radiation sources 14, 16 have moved to the respective positions b and d, according to switching signals for the radiation sources 14, 16, which are supplied from the radiation source switcher 36. The image processor 38 then stores the processed items of radiographic image information in an image memory 40. The items of radiographic image information, which have been stored in the image memory 40, are processed by a tomographic image generator 42 to form a tomographic image of the region 19, which is displayed on a display unit 44.

The tomosynthesis apparatus 10 according to the first embodiment is basically constructed as described above. Operations of the tomosynthesis apparatus 10 according to the first embodiment will be described below.

First, image capturing conditions, including a tube voltage, a tube current, and irradiation times for the radiation X1, X2, which correspond to a region of the subject 18 to be imaged, are set using the image capturing condition setting unit 28. The image capturing conditions are set commonly for the radiation sources 14, 16. The image capturing conditions that have been set are adjusted by the image capturing condition adjuster 30 in order to equalize the doses of radiation X1, X2 that is emitted from the radiation sources 14, 16, using the adjustment data stored in the adjustment data memory 32. For example, a coefficient for adjusting the mAs value of either one of the radiation sources 14, 16 is read as adjustment data from the adjustment data memory 32, and the image capturing conditions are adjusted based on the read adjustment data.

The image capturing conditions set using the image capturing condition setting unit 28, and the image capturing conditions adjusted by the image capturing condition adjuster 30, are supplied to the first radiation source controller 24 and the second radiation source controller 26. When a radiographic technician in charge of the tomosynthesis apparatus 10 presses a shooting switch (not shown), the first radiation source controller 24 and the second radiation source controller 26 control the radiation sources 14, 16 respectively according to the supplied image capturing conditions, thereby causing the radiation sources 14, 16 to emit radiation X1, X2 respectively at equal doses.

When the radiation sources 14, 16 initially begin to emit radiation X1, X2, the radiation sources 14, 16 are located in respective positions a and c. The radiation source synchronous movement controller 34 controls the moving mechanism 12 in order to move the radiation sources 14, 16 synchronously and in respective directions from the positions a and c, toward the positions b and d.

Positional information of the radiation sources 14, 16, as they are moved by the moving mechanism 12, is supplied from the radiation source synchronous movement controller 34 to the radiation source switcher 36. According to the supplied positional information, the radiation source switcher 36 controls the first radiation source controller 24 and the second radiation source controller 26 so as to selectively turn on and off the radiation sources 14, 16. More specifically, the radiation source switcher 36 controls the first radiation source controller 24 and the second radiation source controller 26 in order to repeat a process of turning on the radiation source 14 to emit radiation X1 while simultaneously turning off the radiation source 16, and then turning off the radiation source 14 while simultaneously turning on the radiation source 16 to emit radiation X2. In this manner, radiation X1 and X2, which are alternately emitted from the radiation sources 14, 16, are applied to the subject 18.

Radiation X1, X2 applied to and transmitted through the subject 18 is detected respectively by the radiation detector 20, which converts the radiation X1, X2 into radiographic image information. The radiographic image information generated by the radiation detector 20 is supplied as radiographic image information, at respective positions of the radiation sources 14, 16, to the image processor 38. The radiographic image information then is stored in the image memory 40, according to positional information of the radiation sources 14, 16 supplied from the radiation source synchronous movement controller 34 and the switching signals, which are supplied from the radiation source switcher 36.

When the radiation sources 14, 16 have moved to their respective positions b and d, and all of the radiographic image information generated during movement of the radiation sources 14, 16 to the respective positions b and d has been stored in the image memory 40, one image capturing process is completed. The time required to move the radiation sources 14, 16 during the image capturing process is one-half of the time that would be required if one radiation source were moved from the position c to the position b at the same speed. Therefore, the time required to complete one image capturing process is reduced to one-half, although the acquired amount of radiographic image information remains the same. In other words, assuming that the time required to move the radiation sources 14, 16 during the image capturing process is the same as the time that would be required if only one radiation source were moved from the position c to the position b at the same speed, the acquired amount of radiographic image information is doubled. Accordingly, it is possible to produce a tomographic image having a high level of resolution.

Once the image capturing process is completed, the tomographic image generator 42 reads out and processes the radiographic image information stored in the image memory 40 in order to generate a tomographic image of the region 19 of the subject 18. The tomographic image generator 42 may generate a tomographic image according to a known process, such as a shift-and-add process or a filtered back projection (FBP) process. The generated tomographic image is displayed on the display unit 44 to enable interpretation thereof by a doctor for diagnostic purposes. In this case, since the tomographic image can be generated with a high level of resolution based on a large number of items of radiographic image information, the tomographic image can be interpreted in detail and with high accuracy in order to perform a diagnosis.

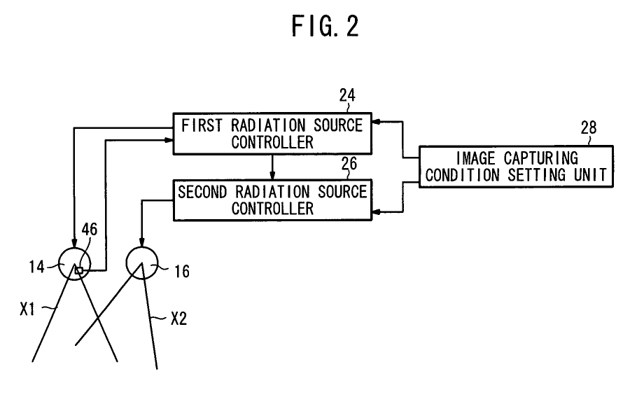

FIG. 2 shows in block form a modification of the tomosynthesis apparatus 10 according to the first embodiment. As shown in FIG. 2, the modified tomosynthesis apparatus includes a dose detector 46 associated with one of the radiation sources 14. The dose detector 46 detects the dose of radiation X1 emitted from the radiation source 14. The dose of radiation X2 emitted from the other radiation source 16 is adjusted based on the detected dose of radiation X1 emitted from the radiation source 14, such that the doses of radiation X1 and X2 emitted from the radiation sources 14, 16 will be equal to each other. Accordingly, there is no need to prepare adjustment data in advance for adjusting image capturing conditions. Furthermore, even if the doses of radiation X1, X2 vary over time, the doses of radiation X1, X2 can be adjusted and maintained equal to each other at all times, in view of such variations. Alternatively, the doses of radiation X1, X2 may be detected and adjusted so as to remain at a constant level.

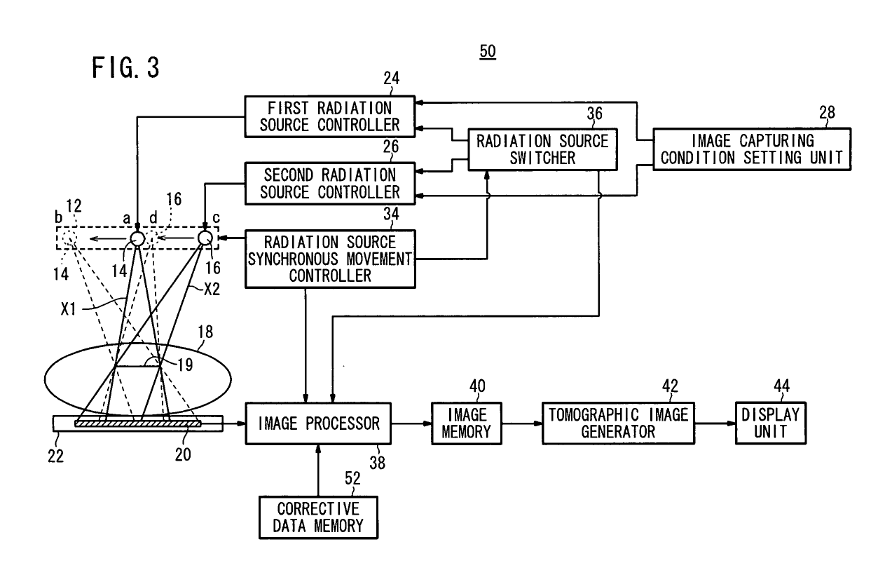

FIG. 3 shows schematically, and partially in block form, a tomosynthesis apparatus 50, which makes up a tomographic image capturing apparatus according to a second embodiment of the present invention. Those parts of the tomosynthesis apparatus 50 which are identical to those of the tomosynthesis apparatus 10 are denoted using identical reference characters, and such features will not be described in detail below.

The tomosynthesis apparatus 50 does not have the image capturing condition adjuster 30 or the adjustment data memory 32, as shown in FIG. 1. Additionally, the tomosynthesis apparatus 50 includes a corrective data memory 52 that stores corrective data for correcting the radiographic image information acquired from the radiation detector 20.

The first radiation source controller 24 and the second radiation source controller 26 control the radiation sources 14, 16 in order to emit and apply radiation X1, X2 respectively to the subject 18 according to common image capturing conditions that are set by the image capturing condition setting unit 28. The image processor (image corrector) 38 corrects the radiographic image information, which is acquired from the radiation detector 20, with corrective data stored in the corrective data memory 52, thereby generating radiographic image information, wherein the difference between the doses of radiation X1, X2 is canceled out. The corrected radiographic image information then is stored in the image memory 40, and thereafter is processed by the tomographic image generator 42 to form a tomographic image of the region 19, which is displayed on the display unit 44 so as to enable the tomographic image to be interpreted by a doctor for diagnosis.

As shown in FIG. 2, at least one of the doses of radiation X1, X2 may be detected, and the radiographic image information may be corrected based on corrective data according to the detected dose.

The present invention is not limited to the aforementioned embodiments. It is a matter of course that various changes and modifications may be made to the embodiments without departing from the scope of the invention as set forth in the appended claims.

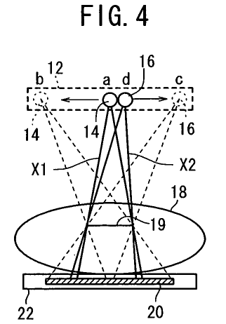

For example, although in the aforementioned tomosynthesis apparatus 10, 50, the radiation sources 14, 16 are moved in the same direction while image capturing is carried out, as shown in FIG. 4, the radiation sources 14, 16 may be arranged to begin moving away from each other synchronously from respective positions a and d which are proximate to each other, so as to separate respectively from one another in the directions indicated by the arrows, and thereafter, one image capturing process is completed when the radiation sources 14, 16 reach respective positions b and c. Since the radiation sources 14, 16 move away from each other synchronously in different directions, vibrations produced by movement of the radiation sources 14, 16 are canceled out. Consequently, the tomosynthesis apparatus is capable of acquiring radiographic image information more accurately and reliably.

What is claimed is:

1. A tomographic image capturing apparatus comprising:
a plurality of radiation sources movable along a predetermined path, for applying respective radiation to a subject at different angles and at different times;
a dose adjuster for adjusting the radiation sources to equalize doses of the radiation emitted from the radiation sources;
a radiation detector for detecting the radiation transmitted through the subject while the radiation sources move along the predetermined path, and converting the detected radiation into respective items of radiographic image information; and
a tomographic image generator for generating a tomographic image of the subject based on the items of radiographic image information converted from the radiation detected by the radiation detector.

2. A tomographic image capturing apparatus according to claim 1, further comprising:
an adjustment data memory for storing adjustment data for adjusting the radiation sources to equalize doses of the radiation emitted from the radiation sources,
wherein the dose adjuster adjusts the radiation sources based on the adjustment data.

3. A tomographic image capturing apparatus according to claim 1, further comprising:
a dose detector for detecting doses of the radiation emitted from the radiation sources,
wherein the dose adjuster adjusts the radiation sources in order to equalize the doses of the radiation, which have been detected by the dose detector.

4. A tomographic image capturing apparatus according to claim 1, wherein the tomographic image capturing apparatus comprises a tomosynthesis apparatus.

5. A tomographic image capturing apparatus comprising:
a plurality of radiation sources movable along a predetermined path, for applying respective radiation to a subject at different angles and at different times;
a radiation detector for detecting the radiation transmitted through the subject while the radiation sources move along the predetermined path, and converting the detected radiation into respective items of radiographic image information;
an image corrector for correcting the items of radiographic image information in order to cancel out a difference between doses of the radiation emitted from the radiation sources; and
a tomographic image generator for generating a tomographic image of the subject based on the items of radiographic image information which have been corrected by the image corrector.

6. A tomographic image capturing apparatus according to claim 5, further comprising:
an adjustment data memory for storing corrective data for correcting the items of radiographic image information in order to cancel out the difference between doses of the radiation emitted from the radiation sources,
wherein the image corrector corrects the items of radiographic image information based on the corrective data.

7. A tomographic image capturing apparatus according to claim 5, wherein the tomographic image capturing apparatus comprises a tomosynthesis apparatus.
